(19) United States
(12) Patent Application Publication
(10) Pub. No.: US 2010/0021543 A1
(43) Pub. Date: Jan. 28, 2010

(54) PERORAL SOLID PAIN KILLER PREPARATION

(75) Inventor: Detlef Schierstedt, St. Augustin (DE)

Correspondence Address: Clements Bernard PLLC, 1901 Roxborough Road, Suite 250, Charlotte, NC, 28211, US

(73) Assignee: KREWEL MEUSELBACH GMBH, Eitorf (DE)

(21) Appl. No.: 12/442,132

(22) PCT Filed: Mar. 27, 2008
(86) PCT No.: PCT/EP07/57024

§ 371 Date: Apr. 23, 2009

(30) Foreign Application Priority Data
Sep. 22, 2006 (DE) 102006044694.1

(51) Int. Cl. A61K 9/14; A61K 31/485; A61K 9/28; A61P 25/04

(52) U.S. Cl. 424/474; 424/484; 514/282; 424/486; 424/488

(57) ABSTRACT

The invention relates to peroral solid analgesic formulations containing opioids and/or morphine analogues in an effect-retarding matrix.

The invention relates to peroral solid analgesic formulations containing opioids and/or morphine analogues in an effect-retarding matrix.

EP 1 327 446 B1 relates among others to controlled release pharmaceutical compositions containing oxycodone. Without a particular definition being needed, oxycodone is known to be a highly effective analgesic. In EP 1 327 446 B1, oxycodone is incorporated in a retarding matrix as an example of a highly effective analgesic, in order that the effect may be sustained in the patient over an extended period of time. According to this document, the matrix causing the retarding effect may be any matrix that yields the in vitro release rates of oxycodone within the narrow ranges required. Preferably, the matrix is a controlled release matrix, although normal release matrices may be used with a coating that controls the release of the active ingredient. Suitable materials for inclusion in a matrix with controlled release include
(a) hydrophilic polymers, such as rubbers, cellulose ethers, acrylic resins and protein-derived materials. Of these polymers, the cellulose ethers, especially hydroxyalkylcelluloses and carboxyalkylcelluloses, are preferred. The oral dosage form may contain from 1% by weight to 80% by weight of at least one hydrophilic or hydrophobic polymer;
(b) cleavable or digestible long-chain (C₈₋₅₀, especially C₁₂₋₄₀) substituted or unsubstituted hydrocarbons, such as fatty acids, fatty alcohols, glyceryl esters of fatty acids, mineral and vegetable oils and waxes. Hydrocarbons having a melting point of from 25° C. to 90° C. are preferred. Of these long-chain hydrocarbon compounds, (aliphatic) fatty alcohols are preferred. The oral dosage form may contain up to 60% by weight of at least one cleavable long-chain hydrocarbon;
(c) polyalkylene glycols. The oral dosage form may contain at least 60% by weight of a polyalkylene glycol.

A particularly suitable matrix comprises at least one water-soluble hydroxyalkylcellulose, at least one aliphatic C₁₂₋₂₃, preferably C₁₄₋₂₂, alcohol and optionally at least one polyalkylene glycol.

Said at least one hydroxyalkylcellulose is preferably a hydroxy(C₁₋₆)alkylcellulose, such as hydroxypropylcellulose, hydroxypropylmethylcellulose and, in particular, hydroxyethylcellulose. The amount of said at least one hydroxyalkylcellulose in the present oral dosage form is determined among others by the exact rate of oxycodone release required. Preferably, however, the oral dosage form contains from 5% by weight to 25% by weight, especially from 6.25% by weight to 15% by weight, of said at least one hydroxyalkylcellulose.

From EP 0 960 619 B1 and the patent references therein, further retarding agents for opioids are known. In particular, the retarding agents described herein are selected from hydrophilic or hydrophobic polymers, i.e., cellulose derivatives, especially alkylcelluloses or hydroxyalkylcelluloses having 1 to 6 carbon atoms in the alkyl or hydroxyalkyl residue.

WO 01/47497 A2 describes pharmaceutical compositions with controlled release of tramadole hydrochloride, which is contained therein in an amount of from 100 to 200 mg. In addition to the actual active ingredient, this composition includes micronized fats, alkali salts of phosphoric acid, non-ionic vinylpyrrolidone polymers, salts of higher fatty acids with alkaline earth metals and silicon oxides.

US 2004/0253310 A1 describes a pharmaceutical composition provided with a coating that is insoluble in aqueous media and has at least one opening, in which the controlled release of the active substance is effected by erosion though the opening contained in the coating.

DE 696 29 797 T2 also describes controlled release pharmaceutical compositions that include, in addition to the actual therapeutically active substance, further pharmaceutically acceptable ingredients, such as a sodium alginate, a water-swellable polymer, an edible C₈-C₅₀ hydrocarbon derivative having a melting point within a range of from 25° C. to 90° C., and a divalent salt selected from the group consisting of an iron salt, a zinc salt, a magnesium salt, an aluminum salt and a calcium salt as well as mixtures of all the above.

In U.S. Pat. No. 3,629,393, tablets with a time-dependent release of the active ingredient are prepared by blending and compressing three different types of granules.

DE 24 04 257 A1 also describes compositions for the retarded release of the active ingredient contained therein which contain, in addition to the latter, mainly metal soaps as bulking agents, polyvinyl acetate as a synthetic resin and cellulose acetate phthalate as a substance causing the sustained release.

As compared to the above mentioned prior art, the object of the present invention is to provide a universally applicable matrix for opioids and/or morphine analogues in various concentrations and dosage forms, especially for oxycodone.

In a first embodiment, the above object is achieved by a peroral solid analgesic formulation containing opioids and/or morphine analogues in an effect-retarding matrix, characterized in that said matrix includes a framework, solid at room temperature, of natural and/or synthetic inorganic calcium salts that are insoluble in water.

By means of the present invention, it is possible to retard the analgesic effect of the mentioned opioids or morphine analogues over a period of time, wherein the surrounding matrix need not necessarily disintegrate upon peroral intake. Said framework, solid at room temperature, of natural and/or synthetic inorganic calcium salts that are insoluble in water as contained according to the invention behaves in a way similar to that of a sponge that slowly releases the opioid and/or morphine analogue contained therein.

More preferably within the meaning of the present invention, any per se known calcium salts that are insoluble in water at room temperature (25° C.) are suitable within the scope of the present invention. “Insoluble” within the meaning of the present invention includes the definition that the salt or compound in question has a solubility of less than 2% by weight in water at room temperature (25° C.).

A particular advantage of the use of frameworks that are solid at room temperature is their proving to be particularly stable towards changes of modification when stored.

More preferably within the meaning of the present invention, the matrix comprises calcium sulfate and/or calcium phosphates, especially monocalcium phosphate, dicalcium phosphate and/or tricalcium phosphate. Accordingly, it is possible within the meaning of the present invention to employ natural and/or synthetic inorganic calcium salts insoluble in water. More preferably within the meaning of the present invention, the amount of inorganic calcium salts employed in the peroral solid analgesic formulations is from 20% by weight to ⁹⁰% by weight, especially from 60% by weight to 70% by weight. The peroral solid analgesic formulations according to the invention preferably contain from 1 to 500 mg, especially from 5 to 40 mg of (at least one of the) opioids and/or morphine analogues, for example, from 5 to 80 mg of oxycodone, which also include the corresponding salts thereof in the matrix. The in vitro release rate of the analgesic formulations is determined by the so-called USP paddle method at 100 rpm in 900 ml of aqueous buffer (for example, artificial intestinal solution at 37° C. in the course of several hours). The USP blade agitator (paddle) method is a blade agitator method as described, for example, in the U.S. Pharmacopeia XII (1990).

In accordance with the prior art, the present invention also relates to the use of the opioids and/or morphine analogues for substantially reducing the range of daily dosages required for pain control in about 90% of the patients.

In combination with the framework, solid at room temperature, of natural and/or synthetic inorganic calcium salts that are insoluble in water, the matrix may alternatively or cumulatively comprise linear or branched, saturated, mono- or polyunsaturated, monovalent or polyvalent, natural or synthetic fatty acids solid at room temperature or their alkaline earth salts each having from 10 to 30 carbon atoms.

“Fatty acids” within the meaning of the present invention may optionally include natural or synthetic mixtures having a range of chain lengths. More preferably within the meaning of the present invention, the fatty acid residues of the matrix are derived from stearic acid, especially magnesium stearate and/or calcium arachinate.

More preferably within the meaning of the present invention, the peroral solid analgesic formulation includes the alkaline earth salts or fatty acids of the mentioned fatty acids in an amount of from 20 to 90% by weight, especially from 60 to 70% by weight. In particular, this quantitative information applies for the case where the mentioned salts are contained as the sole matrix formers (without calcium salts).

Alternatively or cumulatively to the fatty acid salts, their free fatty acids may also be employed as matrix formers as long as the matrix optionally comprises the above mentioned calcium salts. The use of fatty acids is per se known from the above mentioned prior art. However, in the present case, it is particularly preferred to increase the amount of fatty acids as compared to this prior art and to use them as matrix formers, so that from 10 to 20% by weight of the fatty acids and/or fatty acid salts, optionally in combination with from 60 to 70% by weight of the calcium salts, form the matrix in the formulations according to the invention. By analogy, the matrix may also contain from 10 to 20% by weight of the fatty acids in addition to the fatty acid salts in an amount of from 60 to 70% by weight.

In principle, any usual opioids and/or morphine analogues may be employed. More preferably within the meaning of the present invention, these are selected from oxycodone, tramadol, tilidine, morphine, hydromorphone, codeine, hydrocodeine, levorphanol, methadone, meperidine and/or heroine.

Optionally, the formulations according to the invention may also contain per se known morphine antagonists in usual amounts. For example, the use of naloxone, for example, as naloxone hydrochloride semihydrate, naltrexone and methyl-naltrexone is particularly preferred. In addition to the above mentioned components, the controlled release matrix may optionally contain suitable amounts of other materials, for example, diluents, lubricants, water-soluble fillers, binders, granulating aids, colorants, flavorants and glidants that are usual and known in the pharmaceutical art. For influencing the release rate, weakly swellable, especially non-swellable, water-soluble auxiliary agents are desirable, preferably those selected from the groups of nonpolymers and polymers.

More preferably within the meaning of the present invention, polymers are selected from water-soluble polyvinyl alcohols, polyether glycols, such as PEG 2000, 3000, 4000, 10000 and pyrrolidone derivatives, for example, Kollidon® VA64, or polyvinylpyrrolidone, such as Kollidon® 25, 30. More preferred from the group of water-soluble nonpolymers are sugars, mono- and disaccharides, such as sucrose, fructose, glucose, and sugar alcohols, such as sorbitol, xylitol and/or mannitol.

The formulations according to the invention are characterized in that the release rate of the opioids and/or morphine analogues is clearly retarded as compared to a usual standard formulation. As described above, the release rate of the opioids and/or morphine analogues is determined by the USP paddle method in the Examples as well. Accordingly, a formulation is particularly preferred if the release rate of the opioids in the buffer corresponding to artificial intestinal solution, for example, pH 7.2, is

from 12.5 to 42.5% by weight after one hour;

from 25 to 55% by weight after two hours;

from 45 to 75% by weight after four hours; and

from 55 to 85% by weight after six hours.

The formulations according to the invention may be in almost any employable form, for example, as granules, tablets, especially film tablets, coated tablets and/or capsules.

In addition to the ingredients described above, the analgesic formulation may additionally comprise cellulose, because it has an additional retarding effect on the release of the active ingredient. Preferred amounts of cellulose are from 3 to 30% by weight, amounts of from 7 to 13% by weight being particularly preferred. When the proportions of these ingredients are lower, a non-satisfactory retarding effect is observed.

Microcrystalline cellulose, especially Avical®, Avicel®, Emcocel®, Heweten® or Vivapur®, are correspondingly preferred as retarding agents according to the invention.

Further, the analgesic formulation may also include an organic and/or inorganic buffer, preferably in an amount of from 1 to 30% by weight, more preferably in an amount of from 2 to 10% by weight. Namely, much like cellulose, buffers have also shown a drug release retarding effect.

Of such buffers, the salts of citric and/or phosphoric acids, preferably sodium dihydrogenphosphate (NaH₂PO₄), disodium hydrogenphosphate (Na₂HPO₄), sodium dihydrogencitrate (NaC₆H₇O₇), disodium hydrogencitrate (Na₂C₆H₆O₇), have proven particularly favorable, more preferably sodium phosphate (Na₃PO₄) or sodium citrate (Na₃C₆H₅O₇). “Salts” as used herein means not only compounds having the elemental composition stated above, but also usual hydrates and the various modifications thereof.

Further, alternatively or cumulatively, oxides, hydroxides and/or carbonates of alkali and/or alkaline earth metals, for example, magnesium oxide, may also be employed as buffers.

Further, it has been surprisingly shown that cellulose and buffers together have a synergistic effect, since the release is more retarded when a mixture of the two components is used as compared to using exclusively one of the two retarding agents.

In order that the time between the administration and the onsetting effect of the analgesic should not become too long (with the enteric layer alone, the active ingredient would be released only after this layer is dissolved in the small intestine), this combination of matrix and enteric film is advantageously surrounded by a drug-containing layer. Upon entering the stomach, this outermost layer will dissolve immediately and thus provides for an initial dose of the active ingredient.

In addition, this kind of tablet structure may be found particularly useful in cases where the (protonated) active ingredient has a particularly high solubility in acidic medium.

Advantageously, in such formulations, the active Ingredient is distributed in such a way that the effect-retarding matrix contains from 60 to 70% by weight of the total amount of active ingredient, and the drug-containing layer contains from 40 to 30% by weight thereof.

The enteric film preferably comprises shellac, cellulose acetate phthalate (CAP) and/or Eudragit®.

### EXAMPLES

### Example 1

From 80 mg of tramadole hydrochloride, 355 mg of dicalcium phosphate dihydrate (coarse powder), 50 mg of Kollidon® 30 and 70 mg of magnesium stearate, a tablet was prepared by direct pressing (optionally after wetting/drying and pressing). The total weight of the tablet was 555 mg.

The release rates were determined as follows according to the description:

| Time (hours) | tramadole (%) |
|---|---|
| 1 | 39.05 |
| 2 | 53.18 |
| 4 | 69.50 |
| 6 | 81.42 |

### Example 2

From 80 mg of tramadole hydrochloride, 325 mg of dicalcium phosphate dihydrate (fine powder), 10 mg of Kollidon® 30, 40 mg of PEG 3000 and 110 mg of magnesium stearate, a tablet was prepared by direct pressing (optionally after wetting/drying and pressing). The total weight of the tablet was 565 mg.

The release rates were determined as follows according to the description:

| Time (hours) | tramadole (%) |
|---|---|
| 1 | 35.2 |
| 2 | 45.61 |
| 4 | 63.02 |
| 6 | 73.24 |

### Example 3

From 80 mg of tramadole hydrochloride, 325 mg of dicalcium phosphate dihydrate (fine powder), 10 mg of Kollidon® 30, 40 mg of Karion® P 300 and 110 mg of magnesium stearate, a tablet was prepared by direct pressing (optionally after wetting/drying and pressing). The total weight of the tablet was 565 mg.

The release rates were determined as follows according to the description:

| Time (hours) | tramadole (%) |
|---|---|
| 1 | 37.8 |
| 2 | 49.15 |
| 4 | 66.64 |
| 6 | 77.65 |

### Example 4

From 80 mg of tramadole hydrochloride, 325 mg of dicalcium phosphate dihydrate (coarse powder), 50 mg of Kollidon® VA 64 and 100 mg of magnesium stearate, a tablet was prepared by direct pressing (optionally after wetting/drying and pressing). The total weight of the tablet was 555 mg.

The release rates were determined as follows according to the description:

| Time (hours) | tramadole (%) |
|---|---|
| 1 | 34.21 |
| 2 | 45.90 |
| 4 | 61.37 |
| 6 | 71.27 |

### Example 5

From 80 mg of tramadole hydrochloride, 325 mg of dicalcium phosphate dehydrate (coarse powder), 50 mg of Kollidon® 30 and 70 mg of magnesium stearate, a tablet was prepared by direct pressing (optionally after wetting/drying and pressing). The total weight of the tablet was 555 mg.

The release rates were determined as follows according to the description:

| Time (hours) | tramadole (%) |
|---|---|
| 1 | 38.68 |
| 2 | 52.97 |
| 4 | 69.45 |
| 6 | 82.20 |

### Example 6

From 80 mg of tramadole hydrochloride, 355 mg of dicalcium phosphate dehydrate (coarse powder), 50 mg of Kollidon® 30 and 70 mg of stearic acid, a tablet was prepared by direct pressing (optionally after wetting/drying and pressing). The total weight of the tablet was 555 mg.

The release rates were determined as follows according to the description:

| Time (hours) | tramadole (%) |
|---|---|
| 1 | 37.71 |
| 2 | 45.32 |
| 4 | 58.31 |
| 6 | 67.41 |

### Example 7

From 80 mg of tramadole hydrochloride, 355 mg of calcium sulfate dihydrate, 50 mg of Kollidon® 30 and 70 mg of magnesium stearate, a tablet was prepared by direct pressing (optionally after wetting/drying and pressing). The total weight of the tablet was 555 mg.

The release rates were determined as follows according to the description:

| Time (hours) | tramadole (%) |
|---|---|
| 1 | 36.41 |
| 2 | 46.61 |
| 4 | 58.36 |
| 6 | 66.43 |

### Example 8

From 80 mg of oxycodone hydrochloride, 355 mg of dicalcium phosphate dehydrate (coarse powder), 50 mg of Kollidon® 30 and 70 mg of magnesium stearate, a tablet was prepared by direct pressing (optionally after wetting/drying and pressing). The total weight of the tablet was 555 mg.

### Example 9

From 40 mg of oxycodone hydrochloride, 178 mg of dicalcium phosphate dihydrate (coarse powder), 25 mg of Kollidon® 30 and 35 mg of magnesium stearate, a tablet was prepared by direct pressing (optionally after wetting/drying and pressing). The total weight of the tablet was 278 mg.

### Example 10

From 20 mg of oxycodone hydrochloride, 192.45 mg of dicalcium phosphate dihydrate (coarse powder), 27.1 mg of Kollidon® 30, 7.95 mg of magnesium stearate and 30 mg of stearic acid, a tablet was prepared by direct pressing (optionally after wetting/drying and pressing). The total weight of the tablet was 277.3 mg.

The release rates were determined as follows according to the description:

| Time (hours) | oxycodone (%) |
|---|---|
| 1 | 33.2 |
| 2 | 45.8 |
| 4 | 63.2 |
| 6 | 75.0 |

### Example 11

From 10 mg of oxycodone hydrochloride, 192.45 mg of dicalcium phosphate dihydrate (coarse powder), 27.1 mg of Kollidon® 30, 7.95 mg of magnesium stearate and 30 mg of stearic acid, a tablet was prepared by direct pressing (optionally after wetting/drying and pressing). The total weight of the tablet was 267.5 mg.

### Example 12

From 40 mg of oxycodone hydrochloride, 177.5 mg of dicalcium phosphate dehydrate (coarse powder), 25 mg of Kollidon® 30 and 35 mg of stearic acid, a tablet was prepared by direct pressing (optionally after wetting/drying and pressing). The total weight of the tablet was 277.5 mg.

The release rates were determined as follows according to the description:

| Time (hours) | oxycodone (%) |
|---|---|
| 1 | 33.1 |
| 2 | 51.0 |
| 4 | 65.2 |
| 6 | 75.1 |

### Example 13

### Citrate Buffer

From 40 mg of oxycodone hydrochloride, 230.0 mg of dicalcium phosphate dehydrate (coarse powder), 25 mg of Kollidon® 30, 35 mg of stearic acid and 60 mg of trisodium citrate 5.5-hydrate, a tablet was prepared by direct pressing (optionally after wetting/drying and pressing). The total weight of the tablet was 390.0 mg.

### Example 14

### Phosphate Buffer

From 40 mg of oxycodone hydrochloride, 230.0 mg of dicalcium phosphate dehydrate (coarse powder), 25 mg of Kollidon® 30, 35 mg of stearic acid and 30 mg of disodium hydrogenphosphate dihydrate, a tablet was prepared by direct pressing (optionally after wetting/drying and pressing). The total weight of the tablet was 360.0 mg.

### Example 15

### Avicel pH 102

From 40 mg of oxycodone hydrochloride, 230.0 mg of dicalcium phosphate dehydrate (coarse powder), 25 mg of Kollidon® 30, 35 mg of stearic acid and 30 mg of Avicel pH 102, a tablet was prepared by direct pressing (optionally after wetting/drying and pressing). The total weight of the tablet was 360.0 mg.

### Example 16

### Phosphate Buffer +Avicel pH 102

From 40 mg of oxycodone hydrochloride, 230.0 mg of dicalcium phosphate dehydrate (coarse powder), 25 mg of Kollidon® 30, 35 mg of stearic acid, 45 mg of Avicel pH 102 and 15 mg of trisodium phosphate 12-hydrate, a tablet was prepared by direct pressing (optionally after wetting/drying and pressing). The total weight of the tablet was 390.0 mg.

The release rates were determined as follows according to the description:

| Time (hours) | oxycodone (%) |
|---|---|
| 1 | 28.3 |
| 2 | 41.7 |
| 3 | 54.7 |
| 4 | 64.1 |
| 5 | 71.4 |
| 6 | 77.7 |
| 7 | 82.9 |
| 8 | 87.2 |

Example 16 shows that the combination of a microcrystalline cellulose and a buffer has a substantially more pronounced retarding effect as compared to the components alone.

1. A peroral solid analgesic formulation containing opioids and/or morphine analogues in an effect-retarding matrix, characterized in that said matrix includes a framework, solid at room temperature, of natural and/or synthetic inorganic calcium salts that are insoluble in water.

2. The formulation according to claim 1, characterized in that said matrix comprises calcium sulfate and/or calcium phosphates, especially monocalcium phosphate, dicalcium phosphate and/or tricalcium phosphate.

3. The formulation according to claim 1, characterized by comprising the calcium salts in an amount of from 20 to 90% by weight, especially from 60 to 70% by weight.

4. A peroral solid analgesic formulation containing opioids and/or morphine analogues in an effect-retarding matrix, characterized in that said matrix includes linear or branched, saturated, mono- or polyunsaturated, monovalent or polyvalent, natural or synthetic fatty acids solid at room temperature and/or their alkaline earth salts each having from 10 to 30 carbon atoms.

5. The formulation according to claim 4, characterized in that said matrix is derived from stearic acid, magnesium stearate and/or calcium arachinate.

6. The formulation according to claim 4, characterized by comprising the alkaline earth salts or fatty acids respectively in an amount of from 20 to 90% by weight, especially from 60 to 70% by weight.

7. The formulation according to claim 1, characterized in that said matrix comprises the calcium salts and the fatty acid salts and/or their free fatty acids.

8. The formulation according to claim 7, characterized in that said matrix comprises from 60 to 70% by weight of the calcium salts and from 10 to 20% by weight of the fatty acid salts and/or fatty acids.

9. The formulation according to claim 1, characterized in that said opioids and/or morphine antagonists are selected from oxycodone, tramadol, tilidine, morphine, hydromorphone, codeine, hydrocodeine, levorphanol, methadone, meperidine and/or heroine including their salts and bases.

10. The formulation according to claim 1, characterized in that said matrix comprises binders, especially low-viscosity water-soluble polymers or water-insoluble polymers.

11. The formulation according to claim 1, characterized in that said matrix contains water-soluble fillers, especially sugars, sugar alcohols, polyvinyl alcohols and/or pyrrolidone derivatives, especially polyvinylpyrrolidone, or vinylpyrrolidone/vinyl acetate copolymer.

12. The formulation according to claim 1, characterized in that the release rate of said opioids and/or morphine analogues as determined by the USP paddle method is
from 12.5 to 42.5% by weight after one hour;
from 25 to 55% by weight after two hours;
from 45 to 75% by weight after four hours; and
from 55 to 85% by weight after six hours.

13. The formulation according to claim 1, characterized by comprising granules, tablets, especially film tablets, coated tablets and/or capsules.

14. The formulation according to claim 1, characterized by comprising cellulose.

15. The formulation according to claim 14, characterized by comprising cellulose in an amount of from 3 to 30% by weight, especially from 7 to 13% by weight.

16. The formulation according to claim 14, characterized in that said cellulose includes non gel-forming microcrystalline cellulose.

17. The formulation according to claim 1, characterized by comprising an organic and/or inorganic buffer.

18. The formulation according to claim 17, characterized by comprising a buffer in an amount of from 1 to 30% by weight, especially from 2 to 10% by weight.

19. The formulation according to claim 17, characterized by comprising salts of citric and/or phosphoric acids.

20. The formulation according to claim 19, characterized in that said buffer is selected from sodium dihydrogenphosphate (NaH₂PO₄), disodium hydrogenphosphate (Na₂HPO₄), sodium dihydrogencitrate (NaC₆H₇O₇), disodium hydrogencitrate (Na₂C₆H₆O₇), especially sodium phosphate (Na₃PO₄) or sodium citrate (Na₃C₆H₅O₇).

21. The formulation according to claim 17, characterized by comprising oxides, hydroxides and/or carbonates of alkali and/or alkaline earth metals.

22. The peroral solid analgesic formulation containing opioids and/or morphine analogues in an effect-retarding matrix according to claim 1, characterized in that said matrix is surrounded by an enteric film on part or all of its surface, wherein a drug-containing layer is additionally coated on the matrix and/or enteric film.

23. The formulation according to claim 22, characterized in that said effect-retarding matrix contains from 60 to 70% by weight of the total amount of active ingredient, and the drug-containing layer contains from 40 to 30% by weight thereof.
